(19) United States
(12) Patent Application Publication
(10) Pub. No.: US 2023/0372102 A1
(43) Pub. Date: Nov. 23, 2023

(54) CARDIAC ANCHORING SOLUTIONS

(71) Applicant: EDWARDS LIFESCIENCES CORPORATION, Irvine, CA (US)

(72) Inventors: Rohan Yogee Katbamna, Irvine, CA (US); Steven Eric Decker, Anaheim, CA (US)

(21) Appl. No.: 18/361,637

(22) Filed: Jul. 28, 2023

Related U.S. Application Data
(63) Continuation of application No. PCT/US2021/064832, filed on Dec. 22, 2021.
(60) Provisional application No. 63/144,281, filed on Feb. 1, 2021.

(51) Int. Cl. A61F 2/24; A61B 17/04

(52) U.S. Cl. CPC A61F 2/2466; A61F 2/2457; A61B 17/0469; A61B 2017/0409; A61B 2017/0464; A61B 2017/0417; A61B 2017/00044

(57) ABSTRACT

Devices and methods for delivering and deploying cardiac anchors, such as for an atrioventricular heart valve regurgitation reduction implant. An active puncturing tool integrated into an anchor deployment system more accurately insures the location of anchor deployment. One lead of an EKG system connects to a rear end of a puncturing needle which has a conductive tip. Operating the EKG system along with one or both of fluoroscopy and echocardiography systems enables accurate placement of the needle tip. Once located, one or more anchors are deployed from or around the needle. One grappling-hook style anchor has sutures in tension that hold embedded tines from withdrawing from the tissue.

### CROSS-REFERENCES TO RELATED APPLICATIONS

This application is a continuation of International Patent Application No. PCT/US2021/064832, filed Dec. 22, 2021, and titled “Cardiac Anchoring Solutions”, which published as WO 2022/164557, and which claims priority to and the benefit of U.S. Provisional Patent Application No. 63/144,281, filed Feb. 1, 2021, and titled “Cardiac Anchoring Solutions”, the entire disclosures all of which are incorporated by reference for all purposes.

### TECHNICAL FIELD

The present disclosure relates generally to devices and methods for anchoring systems such as an atrioventricular regurgitation prevention system in a ventricle.

### BACKGROUND

Heart valve disease, such as valve regurgitation, is typically treated by replacing or repairing the diseased valve during open-heart surgery. However, open-heart surgery is highly invasive and is therefore not an option for many patients. For high-risk patients, a less-invasive method for repair of heart valves is considered generally advantageous. In patients with severe/torrential tricuspid valve regurgitation, the tricuspid valve annulus and the right ventricle are often seen to have dilated abnormally large amounts, often times resulting in a severe loss of tricuspid valve leaflet coaptation.

One solution is seen in the FORMA Transcatheter Tricuspid Repair System from Edwards Lifesciences, Inc. of Irvine, CA, as well as solutions disclosed in U.S. Pat. No. 9,474,605, both expressly incorporated herein, which introduce a gap-filling element into the tricuspid valve that restores leaflet coaptation, reduces tricuspid regurgitation (TR) and right atrium (RA) pressure, and thereby alleviates classic TR patient symptoms and improves quality of life. A flexible rail having a ventricular anchor on the distal end thereof adapted to anchor into tissue within a ventricle is first deployed percutaneously. A repair catheter passes along the flexible rail, and a leaflet coaptation member or spacer on a distal end of the catheter is located within the native valve leaflets. When in place, the spacer fills gaps between the tricuspid leaflets and reduces or eliminates regurgitation through the native valve. Various alternative anchoring techniques include deployment of the anchor trans-pericardial (through the base of the RV and through the pericardium) and trans-septal (through the interventricular septum from the RV through to the LV). Both U.S. Pat. No. 9,474,605 and WO2020197854A1 document alternative anchoring techniques and are expressly incorporated herein.

Despite these and other cardiac implants anchored in subvalvular spaces, the task of securely anchoring in the ventricles, especially while the heart is beating, remains difficult and requires improvements.

### SUMMARY

The present invention relates generally to devices and methods for securely placing an anchor in the ventricles for a cardiac implant system.

One embodiment disclosed herein is an active puncturing tool in the form of a modified needle apparatus that incorporates EKG-based myocardial puncture sensing to guide in-vivo myocardial puncture in two modes—from the ventricle to the exterior of the heart, and between the ventricles across the septal wall. The modified needle apparatus and delivery system incorporates standard, off-the-shelf 5-lead EKG terminals and monitors (available readily at all hospitals) to add an additional real-time indication of needle tip location within the heart. Used in tandem with fluoroscopy and echocardiography (contrast/agitated saline injections), this dramatically reduces the ambiguity with respect to needle tip visualization, reduces procedure times and reduces intra-procedural complications associated with myocardial puncture.

For trans-pericardial anchoring, the modified needle apparatus and attendant methodology is used to determine what the needle tip is in contact with, specifically for the purpose of bringing the needle tip from the right ventricle out of the heart and into the space between the pericardium and the chest wall. The location of the needle tip is determined as it advances through various zones from observing the EKG trace: namely, in ventricular free space/blood, in contact with the inner wall of the base of the right ventricle, partially through the right ventricular myocardium (lodged inside the myocardium), through the myocardium and in the pericardial space, fully through the pericardium and into the space between the pericardium and the inner chest wall. The EKG traces associated with each of these spaces are logged and each are seen to have distinct traces that can be used to guide RV myocardial puncture.

Alternatively, for trans-septal puncturing and anchoring, the modified needle apparatus and methodology is used to determine primarily when the needle tip crosses over from the RV into the LV. Discrete EKG signals noted here are found when the location of the needle tip advances through various zones as follows: in right ventricular free space/blood, in contact with the inter-ventricular septum myocardium, partially through or wedged inside the inter-ventricular septum myocardium, completely through the inter-ventricular septum and in LV free space, and when advanced too far to be in contact with the LV free wall.

The present application discloses a system for delivering and deploying cardiac anchors, comprising an active puncturing tool. The tool includes a proximal control handle with a flexible sheath extending distally therefrom. A flexible puncturing needle extends through the sheath and is linearly movable therein to a position beyond a distal tip of the sheath, the needle being electrically insulated except at a sharp distal tip. A cardiac anchor is movable through the sheath and relative to the needle to a position beyond the sharp distal tip. Finally, an EKG system is connected to the tool so that one lead is in electrical contact with a proximal end of the needle.

The cardiac anchor delivery and deployment system may further comprise a regurgitation reduction spacer sized to fit within leaflets of an atrioventricular valve and configured to coapt against the leaflets to reduce regurgitation therebetween. The spacer preferably has a length such that the proximal end resides within the atrium and the distal end resides within the ventricle. A flexible tether connects the spacer to the cardiac anchor. In one form, the cardiac anchor is an expandable disk-shaped anchor configured to abut cardiac tissue. In another form, the cardiac anchor is a tissue anchor configured to embed in cardiac tissue.

On tissue anchor configured to embed in cardiac tissue comprises:
a. a tubular barrel defining a longitudinal axis having a plurality of distally-extending tines configured to be embedded into tissue, the tines being biased toward a relaxed configuration where the tines splay radially outward from the axis;
b. a flexible proximal shaft connected to the barrel; and
c. a plurality of sutures each connected to one of the tines and extending proximally through the shaft, each tine extending outward from the barrel and along a respective tine to be fastened at a distal tip thereof, wherein tension on the sutures helps prevent the tines from bending toward the axis upon application of proximal forces on the anchor tending to pull the anchor from within tissue.

In the systems described above, the control handle may have a first slider movable thereon configured to axially displace the needle relative to the sheath, and a second slider movable thereon configured to axially displace the cardiac anchor relative to the needle. The system first and second sliders may be coupled for common movement, and further including a lock which may be released to permit the second slider to move with respect to the first slider. Each of the first and second sliders may have an outer finger tab labeled with an indicator of the respective function of each. The control handle may further include an actuator for angling a tip of the sheath.

In one embodiment, the needle is hollow and the cardiac anchor is positioned within and deployable from within the needle. The control handle may further include a plurality of fluid ports connected thereto for introducing or withdrawing fluid or gas from concentric spaces within the system, including a space between the sheath and needle, and a space between the needle and cardiac anchor. The EKG may be a 5-lead EKG.

Another aspect described herein is a tissue anchor for medically implanted systems, comprising a tubular barrel defining a longitudinal axis having a plurality of distally-extending tines configured to be embedded into tissue, the tines being biased toward a relaxed configuration where the tines splay radially outward from the axis. A flexible proximal shaft connects to the barrel, and a plurality of sutures are each connected to one of the tines and extend proximally through the shaft. Each suture extends outward from the barrel and along a respective tine to be fastened at a distal tip thereof, wherein tension on the sutures helps prevent the tines from bending toward the axis upon application of proximal forces on the anchor tending to pull the anchor from within tissue. Each of the tines may be formed as a laser-cut portion of a tube that also forms the tubular barrel, and each tine has a plurality of cleats along its length through which the sutures pass before reaching the distal tip.

The present application discloses a method for delivering and deploying a cardiac anchor into a patient. The method comprises first providing an active puncturing tool having a proximal control handle with a flexible sheath extending distally therefrom. A flexible puncturing needle extends through the sheath and is linearly movable therein to a position beyond a distal tip of the sheath, the needle being electrically insulated except at a sharp distal tip. A cardiac anchor is movable through the sheath and relative to the needle to a position beyond the sharp distal tip. Also provided is an EKG system, the method involving connecting the EKG system to the patient and connecting one lead to a proximal end of the needle. The sheath is advanced through the vasculature until the distal tip thereof is in proximity to a tissue surface within the heart. The needle is then advanced from the distal tip of the sheath while monitoring a location of the sharp distal tip of the needle on a monitor of the EKG system. Advancement of the needle is halted at a desired location, and the cardiac anchor advanced from within the needle to deploy the cardiac anchor at the desired location.

The method may also include deploying a regurgitation reduction spacer within leaflets of an atrioventricular valve configured to coapt against the leaflets to reduce regurgitation therebetween. Desirably, the spacer has a length such that the proximal end resides within the atrium and the distal end resides within the ventricle.

One such method of deploying a regurgitation reduction spacer includes advancing the sheath until the distal tip thereof is in a cavity of the ventricle in proximity to an inner surface thereof, advancing the needle through cardiac tissue to a bodily space, advancing the cardiac anchor from within the needle, the cardiac anchor being self-expandable to provide an external anchor, withdrawing the needle and sheath, and connecting a flexible tether between the cardiac anchor and the spacer. The cardiac anchor may be an expandable disk-shaped anchor, and the cardiac tissue may be myocardium with the desired location being outside of the heart, for instance outside of the pericardial sac. The ventricle may be a first ventricle, and the cardiac tissue is septal tissue between the first ventricle and a second ventricle, and the desired location is in the second ventricle.

A second such method of deploying a regurgitation reduction spacer includes advancing the sheath until the distal tip thereof is in a cavity of a ventricle in proximity to an inner surface thereof, advancing the needle into cardiac tissue, advancing the cardiac anchor from within the needle, the cardiac anchor being self-expandable to provide an internal tissue anchor embedded in cardiac tissue, withdrawing the needle and sheath, and connecting a flexible tether between the cardiac anchor and the spacer.

In any of the methods described above, the EKG is a 5-lead EKG and the method includes applying 4 of the leads to the chest of the patient. The methods may further include, while advancing the needle, simultaneously monitoring the location of the sharp distal tip of the needle with at least one of fluoroscopy and echocardiography. Similarly, the methods may also include, while advancing the needle, simultaneously monitoring the location of the sharp distal tip of the needle with fluoroscopy and injecting contrast medium into an access tube in the handle which is in fluid communication with an opening at the sharp distal tip of the needle.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of embodiments of the present disclosure, a more particular description of the certain embodiments will be made by reference to various aspects of the appended drawings. It is appreciated that these drawings depict only typical embodiments of the present disclosure and are therefore not to be considered limiting of the scope of the disclosure. Moreover, while the figures may be drawn to scale for some embodiments, the figures are not necessarily drawn to scale for all embodiments. Embodiments of the present disclosure will be described and explained with additional specificity and detail using the accompanying drawings.

FIG. 1 is a schematic view of the final configuration of a prior art percutaneous heart valve regurgitation reduction system having a coapting element or spacer positioned within the tricuspid valve and a proximal length of the repair catheter including a locking collet shown exiting the subclavian vein to remain implanted subcutaneously;

FIGS. 2A and 2B are sectional views of the right side of the human heart showing a spacer for a regurgitation reduction system anchored in the right ventricle with an expandable disk-shaped anchor on the end of an anchoring suture, and FIG. 2C is a detailed view of the expandable anchor;

FIGS. 3A-3C illustrate layers of the heart pierced by an active puncturing tool when deploying the disk-shaped anchor of FIG. 2C, with FIG. 3D showing placement outside of the pericardial sac, and FIG. 3E showing placement in a space between the pericardial sac and the exterior of the myocardium;

FIG. 4 is a diagram of a person's chest cavity showing the location of the heart, and FIG. 4A is enlarged cross-sectional view showing the relationship between the apex of the heart, the pericardial sac, and the surrounding anatomy;

FIG. 5 is a schematic view of a patient showing one arrangement of the active puncturing tool and EKG monitor and electrode placement associated therewith;

FIG. 6 is a perspective view of an exemplary active puncturing tool of the present application, and FIG. 6A shows several outer covers exploded therefrom;

FIG. 7A is an elevational view of the active puncturing tool, FIG. 7B shows several outer covers exploded therefrom, and FIG. 7C is an enlargement of the control handle showing labels on a pair of sliders;

FIGS. 8A and 8B are partial views of the active puncturing tool in several stages of deployment;

FIG. 9 is an enlarged perspective view of the distal end of a puncturing needle showing insulation thereon;

FIGS. 10A/10B, 11A/11B and 12A/12B show gradual advancement of the puncturing needle through the heart wall alongside complementary images of a readout of an EKG system having one electrode connected to the puncturing needle;

FIG. 13 is a perspective view of the active puncturing tool having an alternative anchor;

FIG. 14 is a perspective view of the alternative anchor of FIG. 13, and FIG. 14A is a sectional view therethrough showing activation of a retention system;

FIG. 15 is a view of a puncturing needle of the active puncturing tool embedded in tissue with the alternative anchor of FIG. 14 advancing into the tissue;

FIGS. 16A and 16B are cross-sectional views illustrating two stages in embedding and retaining the alternative anchor into tissue;

FIG. 17A is a cross-sectional view illustrating a conventional anchor embedded into tissue, and FIG. 17B shows the potential for withdrawal from within the tissue from tensile forces;

FIG. 18 is a schematic view showing advancement of an annuloplasty band deployment sheath to a heart valve annulus; and

FIG. 19A is a sectional view illustrating a step of deployment of the annuloplasty band using the sheath of FIG. 19 that incorporates an active puncturing needle as described herein, and FIG. 19B is a perspective view after a portion of the annuloplasty band has been implanted at the annulus.

### DETAILED DESCRIPTION OF THE PREFERRED EMBODIMENTS

The following description refers to the accompanying drawings, which illustrate specific embodiments. Other embodiments having different structures and operation do not depart from the scope of the present disclosure.

The present application discloses systems and methods for anchoring cardiac implants, in particular as illustrated a heart valve regurgitation reduction spacer within a heart valve. Such heart valve regurgitation reduction systems may be implanted within the left or right side of the heart and may extend out of the heart into the vasculature, for example, to the subclavian vein. However, the principles disclosed herein for anchoring such an implanted device are suitable for other applications as well.

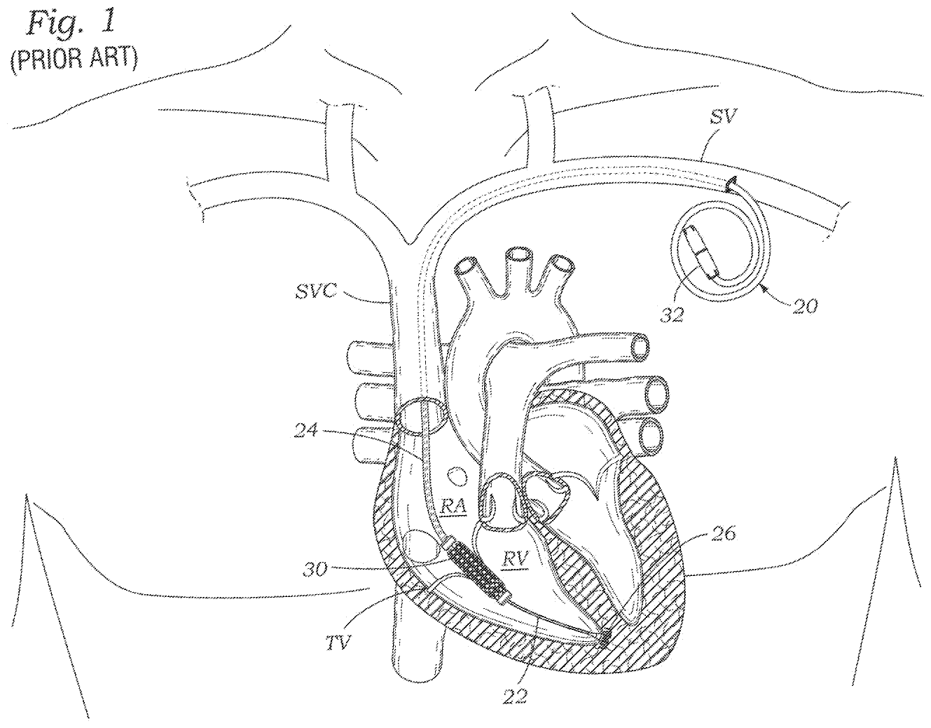

FIG. 1 is a schematic view of the final implanted configuration of a prior art percutaneous heart valve regurgitation reduction system having a coapting element or spacer positioned within the tricuspid valve and a proximal length of the repair catheter including a locking collet shown exiting the subclavian vein to remain implanted subcutaneously. The system includes a repair catheter 20 percutaneously delivered into the right side of the heart to reduce tricuspid valve TV regurgitation. The repair catheter 20 enters the right atrium RA from the superior vena cava SVC after having been introduced to the subclavian vein SV using well-known methods, such as the Seldinger technique. The repair catheter 20 preferably tracks over a smaller diameter pre-installed anchor rail 22 that has also been inserted into the subclavian vein SV and steered through the vasculature until it resides and is anchored at or near the apex of the right ventricle RV, as shown. The repair catheter 20 includes an elongated hollow shaft 24 that may be reinforced, for example, with an embedded braided or coiled structure.

A distal device anchor 26 secures a distal end of the rail 22 at the apex of the right ventricle RV, or to other anatomical features within the ventricle. The anchor rail 22 may be constructed as a braided wire rod, or cable, and is desirably hollow to enable passage over a guide wire (not shown). Further details of the anchor rail 22 and device anchor 26 are seen in U.S. Pat. No. 9,474,605 to Rowe, et al.

The repair catheter shaft 24 carries a coapting element or spacer 30 on its distal end portion that is ultimately positioned within the tricuspid valve TV, the leaflets of which are shown closed in systole and in contact with the spacer 30. A variety of coapting elements may be utilized, the common feature of which is the goal of providing a plug of sorts within the heart valve leaflets to mitigate or otherwise eliminate regurgitation. In the illustrated embodiment, the spacer 30 includes an expandable body formed of a latticework of struts arranged to be auxetic, or have a negative Poisson's ratio, that may be adjusted in vivo, such as disclosed in U.S. Patent Publication No. 2019/0358029, while other coapting elements are disclosed in U.S. Pat. Nos. 9,474,605 and 9,636,223, the entire disclosures which are expressly incorporated herein by reference. The spacer 30 is delivered in a radially contracted state to reduce the size of the incision used and facilitate passage through the vasculature and is then expanded within the valve leaflets.

A locking mechanism is provided on the regurgitation repair catheter 20 to lock the axial position of the spacer 30 within the tricuspid valve TV and relative to the fixed anchor rail 22. For example, a locking collet 32 along the length of the repair catheter shaft 24 permits the physician to selectively lock the position of the shaft, and thus the connected spacer 30, along the anchor rail 22. There are of course a number of ways to lock a catheter over a concentric guide rail, and the application should not be considered limited to the illustrated embodiment. For instance, rather than a locking collet 32, a crimpable section such as a stainless-steel tube may be included on the repair catheter shaft 24 at a location near the skin entry point and spaced apart from the location of the spacer 30. The physician need only position the spacer 30 within the leaflets, crimp the catheter shaft 24 onto the anchor rail 22, and then sever both the catheter and rail above or proximal to the crimp point.

A proximal length of the repair catheter 20 including the locking collet 32 exits the subclavian vein SV through a sealed puncture and remains implanted subcutaneously; preferably coiling upon itself as shown. In the procedure, the physician first ensures proper positioning of the spacer 30 within the tricuspid valve TV, locks the repair catheter 20 with respect to the anchor rail 22 by actuating the locking collet 32, or by another means, and then severs that portion of the repair catheter shaft 24 that extends proximally from the locking collet. The collet 32 and/or coiled portion of the repair catheter shaft 24 may be sutured or otherwise anchored in place to subcutaneous tissues outside the subclavian vein SV. It is also worth noting that because the repair catheter 20 initially slides with respect to the anchor rail 22, it may be completely removed to withdraw the spacer 30 and abort the procedure during implantation. The implant configuration is like that practiced when securing a pacemaker with an electrode in the right atrium muscle tissue and the leads extending to the associated pulse generator placed outside the subclavian vein. Indeed, the current procedure may be performed in conjunction with the implant of a pacing lead.

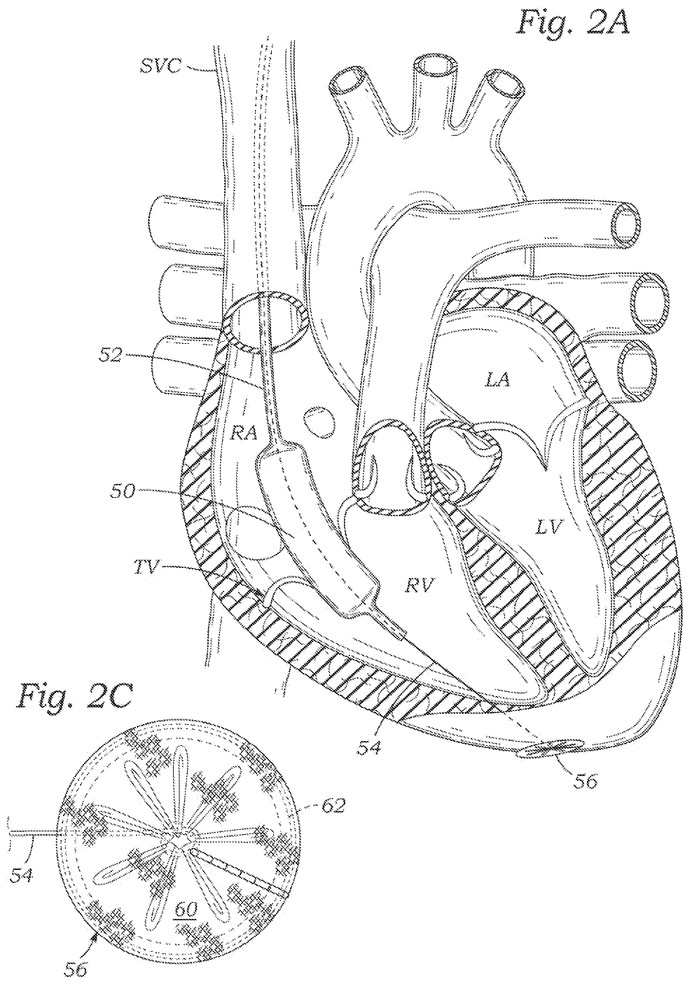

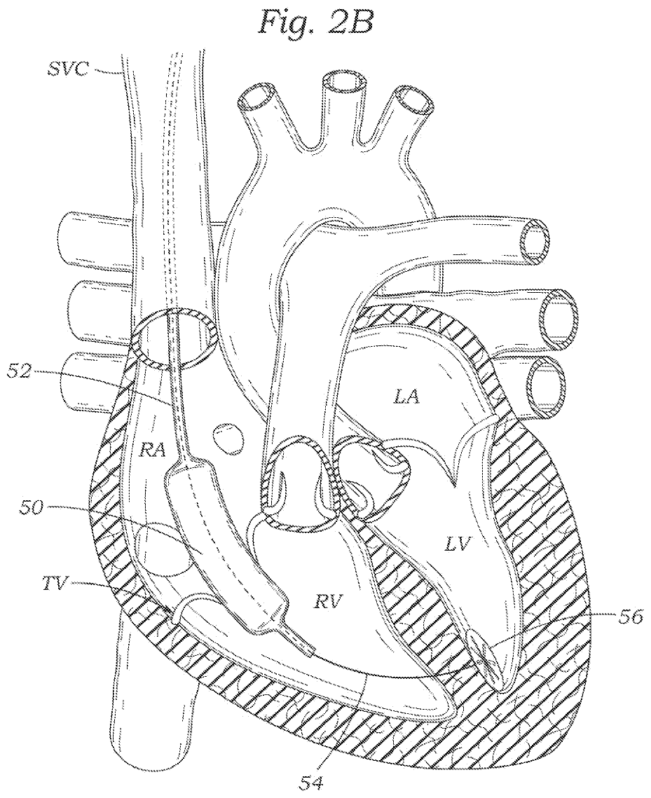

FIG. 2A is a sectional view of the right side of the human heart showing a spacer 50 for a regurgitation reduction system anchored in the right ventricle, with a proximal sheath or shaft 52 connected thereto and extending proximally out of the right atrium RA. The spacer 50 may be anchored on the subvalvular side using an anchoring tether 54 and a tissue anchor 56 in the form of an expanded flat disk positioned exterior to the right ventricle RV.

The anchor 56 could alternatively be placed across the intraventricular septum or septal wall SW, as in FIG. 2B, such that the anchor is deployed in the left ventricle LV and pulled against the LV side of the septum to anchor the spacer 50 within the tricuspid valve TV.

As seen in FIG. 2C, the exemplary disk-shaped anchor 56 comprises a fabric cover 60 having an internal support ring 62 arranged in a circle around a periphery of the anchor 56. The support ring 62 couples to the cover 60, such as with sutures, and is desirably made of a material that is flexible, such that the ring may move from a linear configuration to the ring-shaped configuration shown in FIG. 2C. In one embodiment, the support ring 62 is made of a super elastic or shape-memory material, such as Nitinol, and is shape set to automatically move from the linear configuration for delivery through an access device to the relaxed ring-shaped anchoring configuration. One such expandable anchor is disclosed in U.S. Patent Publication No. 2020/0069426, the entire disclosure of which is expressly incorporated herein by reference. Although a disk-shaped anchor 56 is shown, the present method of locating the anchor prior to deployment is useful for other anchors, expandable or otherwise, and the disclosure should not be limited to the illustrated anchor.

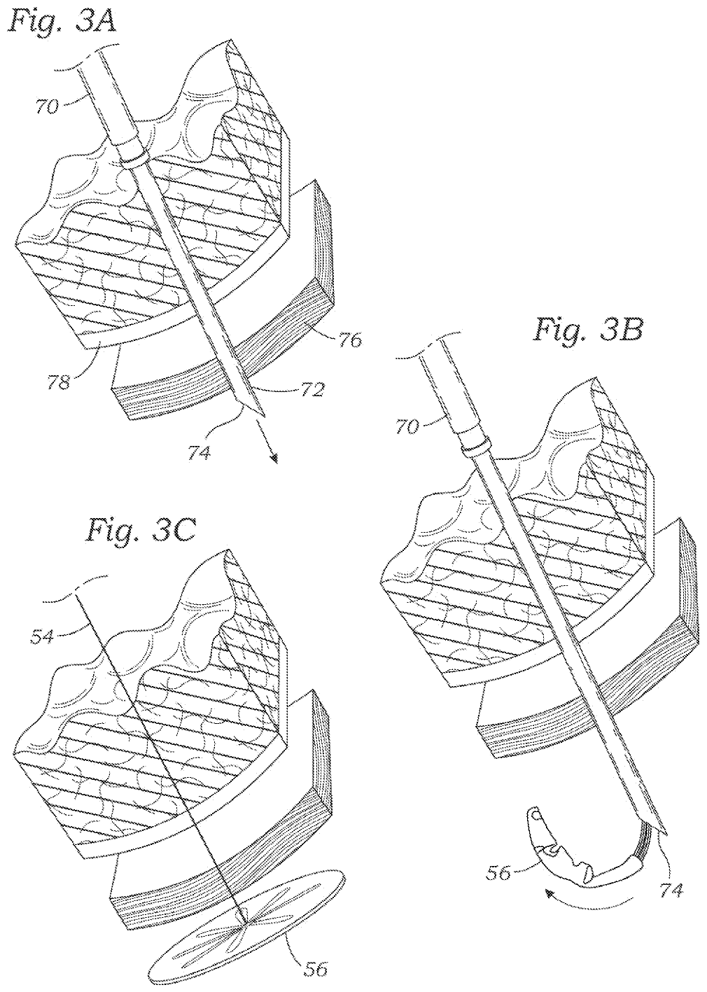

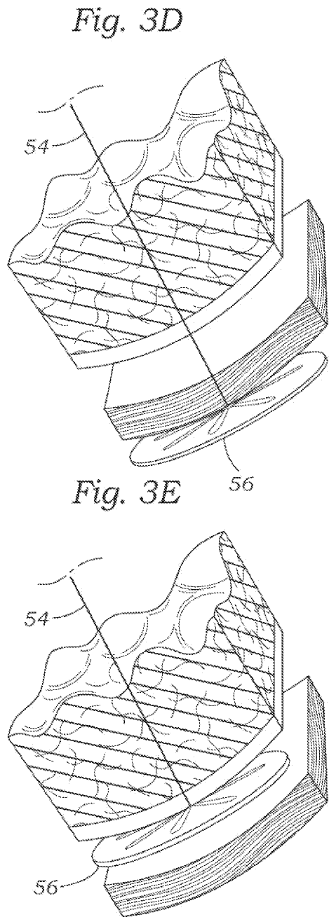

FIGS. 3A-3C illustrate layers of the heart pierced by an active puncturing tool 70 when deploying the disk-shaped anchor 56 of FIG. 2B, with FIG. 3D showing placement outside of the pericardial sac, and FIG. 3E showing placement in a space between the pericardial sac and the exterior of the myocardium. The active puncturing tool 70 includes a puncturing needle 72 which is hooked up as an electrode of an EKG monitoring system, and sends back input to help determine where the tip of the needle is located.

FIGS. 3A-3C show the disk-shaped anchor 56 is initially in a straightened or linear configuration and passes through a lumen of the flexible puncturing needle 72 having a sharpened distal point. For example, the needle 72 may have an angled open distal end 74 from which the disk-shaped anchor 56 in a linear configuration is expelled. Initially, the puncturing needle 72 passes through the myocardium and outer myocardial layer 78 of the heart wall. FIG. 3A shows further advancement of the puncturing needle 72 through to the outside of a pericardial sac 76 which surrounds the heart. In FIG. 3B, the expandable tissue anchor 56 is seen emerging from the distal end 74 to the exterior of the pericardial sac 76. Finally, in FIG. 3C the tissue anchor 56 is shown fully expanded and connected to the tether 54, which may be a suture.

In this trans-pericardial procedure, as will be described in more detail below, the active puncturing tool 70 indicates when the needle tip is still inside the catheter, in contact with the myocardium, within the myocardium, completely through the myocardium and within the space inside the pericardial sac 76, in contact with the pericardium, and through the pericardium. Each of these positions of the needle have an easily identifiable EKG trace.

The tissue anchor 56 is thus delivered to a deployment site in a linear configuration within the tool 70, and then expelled from the distal opening 74 as seen in FIG. 3B. As the tissue anchor 56 is being expelled, tension on the tether 54 acts on the internal support ring 62 to cause it to curl, and eventually the cover 60 and support ring 62 assume the disc-shaped configuration of FIG. 3C, with the tether 54 extending proximally from the cover 60 at the central axis. Retraction of the tether 54 as in FIG. 3D brings the tissue anchor 56 into contact with the exterior of the pericardial sac 76. When expanded, the tissue anchor 56 may have a diameter of between about 20-25 mm, although other diameters may be utilized as desired. The puncturing needle 72 is withdrawn back through the heart wall, and the tether 54 used to anchor to the regurgitation reduction spacer 50, as in FIG. 2A.

Visualization of the location of the puncturing needle 72 while passing through the myocardium and pericardium is challenging solely using fluoroscopy or echocardiography (ultrasound) techniques. Most cath-labs and hybrid operating rooms are equipped with fluoroscopy as well as multi-modal echocardiographic technology, as available imaging modalities which are widely used for many different medical device procedures. Fluoroscopy to visualize contrast bolus injections down the length of the needle and evaluate needle tip location is helpful but provides only an approximate location and is easy to be fooled into a false plane of attack due to a bad fluoroscopy angle. Also, depending on the patient, image quality is not always crisp and contrast bolus injections can often pool in one position or wash away too quickly. Echocardiography is hindered primarily due to the depth of the needle within the heart. Both the trans-pericardial and trans-septal approaches perform punctures deep in the RV, making it difficult for both Transesophageal Echocardiogram (TEE) and Intracardiac ECHO (ICE) to have the relevant resolution needed to fully confirm catheter tip location quickly.

Supplementing one or both of fluoroscopy and echocardiography with the currently disclosed active puncturing tool 70 provides a non-visual confirmation that the needle tip is in contact with the proper location and a secondary real-time indication of what the needle is in contact with. The active puncturing tool 70 is desirably not a replacement for any of these imaging modalities, it is meant to augment and be used in tandem with them for best results with respect to navigating, imaging and controlling myocardial puncture both trans-pericardial and trans-septal passages (and perhaps also for punctures in other places in the heart).

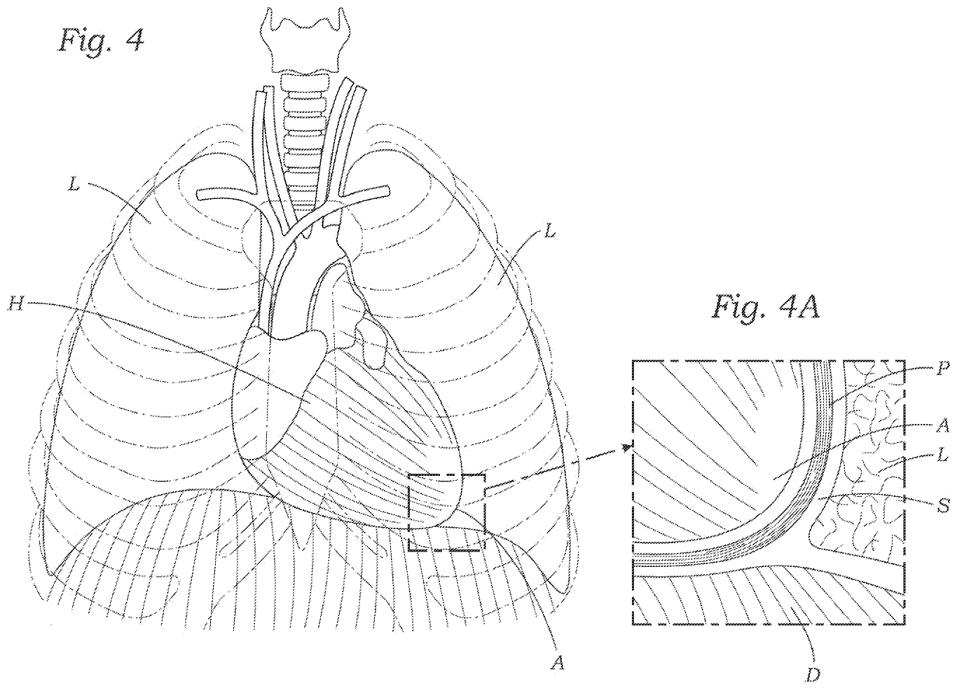

FIG. 4 is a diagram of a person's chest cavity showing the location of the heart H, and FIG. 4A is enlarged cross-sectional view showing the relationship between the apex A of the heart, the pericardial sac P, and the surrounding anatomy. Specifically, the heart H is in the center of the thoracic cavity, medially between the two lobes of the lungs L, and is oriented obliquely, with the apex A pointing down and to the left (from the patient's perspective). The heart H is suspended within a tough fibrous sac, the pericardium P, by its connections to the great vessels: the superior and inferior venae cavae, the pulmonary artery and veins, and the aorta. The pericardium P is fused to the diaphragm D, and so downward movement of the diaphragm during inspiration of the lungs L pulls the heart into a more vertical orientation.

For the trans-pericardial anchoring approach shown above in FIGS. 3A-3D, the needle 72 is advanced through the base of the right ventricle, through the pericardium and into a space S between the outer surface of the pericardium P and the inner surface of the chest wall. Or, the procedure may locate the needle and anchor 56 between the myocardium and pericardium P, as in FIG. 3E. Once punctured, it is critical that the distal anchor 56 is delivered and seated against the pericardium P (or myocardium) to minimize both blood entry into the pericardium (pericardial tamponade) and accumulation of blood in the pleural cavity (hemothorax). These are certain critical risks with the trans-pericardial approach that may be reasonably mitigated by clear identification of the needle tip 72. For instance, due care must be exercised to avoid puncturing the lung L. However, due to limitations of conventional imaging (fluoroscopy, echocardiography) location of the tip of the needle 72 is not always so straightforward. These constraints can cost the procedure precious seconds that would otherwise be spent rapidly deploying the anchor and achieving hemostasis to prevent the aforementioned failure modes.

The needle apparatus described herein also provides advantages for securing the trans-septal anchor as shown in FIG. 2B, as it allows for a better controlled and thus quicker inter-ventricular septal puncture with minimal risk to the patient as a result of overpuncture. The needle is advanced through the bottom third of the RV septal wall and into the LV. Once punctured, the anchor 56 is deployed through the needle and stabilized against the LV septal wall. The primary concern here is that the needle extends too far into the LV and punctures the LV free wall, resulting in unintended complications such as pericardial tamponade or hemothorax, likely requiring surgical intervention immediately to stabilize the patient. The procedure works just as described in the above trans-pericardial approach, and indicates when the needle tip is still inside the catheter, in contact with interventricular septum, within the interventricular septum, completely through the interventricular septum, and in contact with the LV free wall. Each of these positions of the needle have an easily identifiable EKG trace.

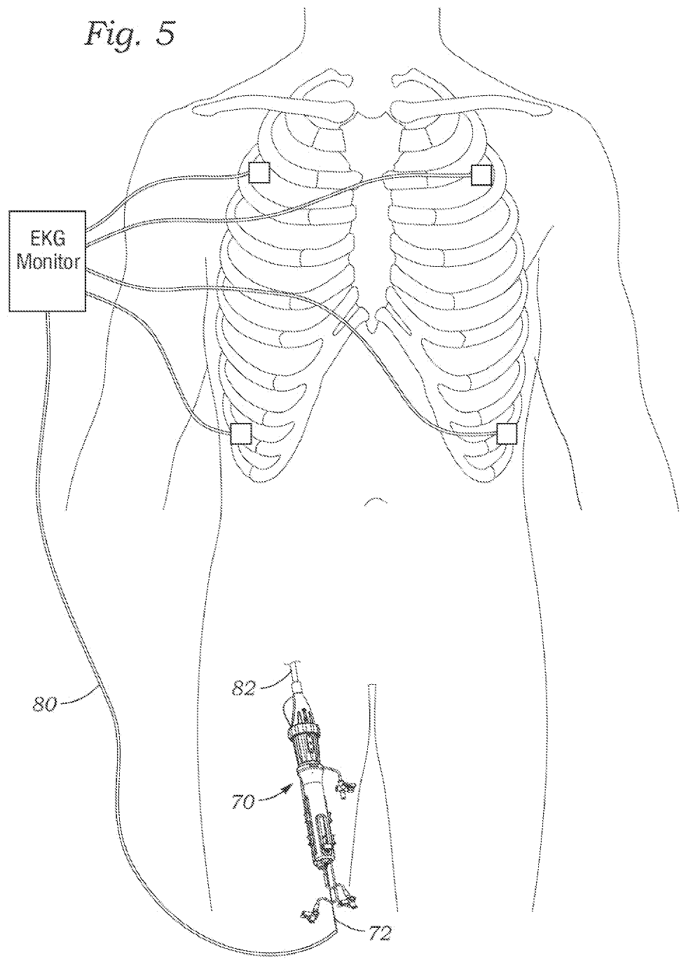

FIG. 5 is a schematic view of a patient showing one arrangement of the active puncturing tool and EKG monitor and electrode placement associated therewith. The active puncturing tool 70 described herein is a custom needle apparatus and anchor delivery system that incorporates widely available 5-lead EKG terminals and an EKG monitor to have a live, real-time readout of what the puncture needle is in contact with (blood, myocardium, pericardium, etc.).

A typical 5-lead EKG setup includes one lead for each of the limbs (2 arms and 2 legs means 4 leads total on the patient) placed on the chest, roughly as seen in FIG. 5. The fifth lead “V” is typically placed on a terminal near the heart, but for the purposes of this invention, the fifth lead 80 is directly attached to a rear end of the active needle tool 70. Additionally, the active needle tool 70 has a custom design which facilitates interaction with the lead and accurate 1:1 transmission of the contact signal between the distal tip of the needle 72 and the monitor, with minimal losses. More particularly, the lead 80 terminates in a coupler such as a clip 81 which securely attaches to a rear end of the needle 72 in a manner which reduces losses.

In the approach technique shown, an elongated flexible sheath 82 of the active needle tool 70 is advanced from the groin area upward through the femoral vein into the right atrium, as will be shown. Techniques for incising the patient and introducing the sheath 82 into the femoral vein are well known, as are flexible sheaths and needles 72 of sufficient length to reach the heart from the groin area. The sheath 82 may be formed of a suitable flexible polymer, while the flexible puncturing needle 72 may be polymeric or Nitinol. The specifics will not be further detailed. Of course, this schematic illustration is but one possible access pathway. Likewise, alternatives pathways to access the right atrium include downward from the neck through the internal jugular vein or subclavian vein, and the present application should not be considered limited to any particular access pathway.

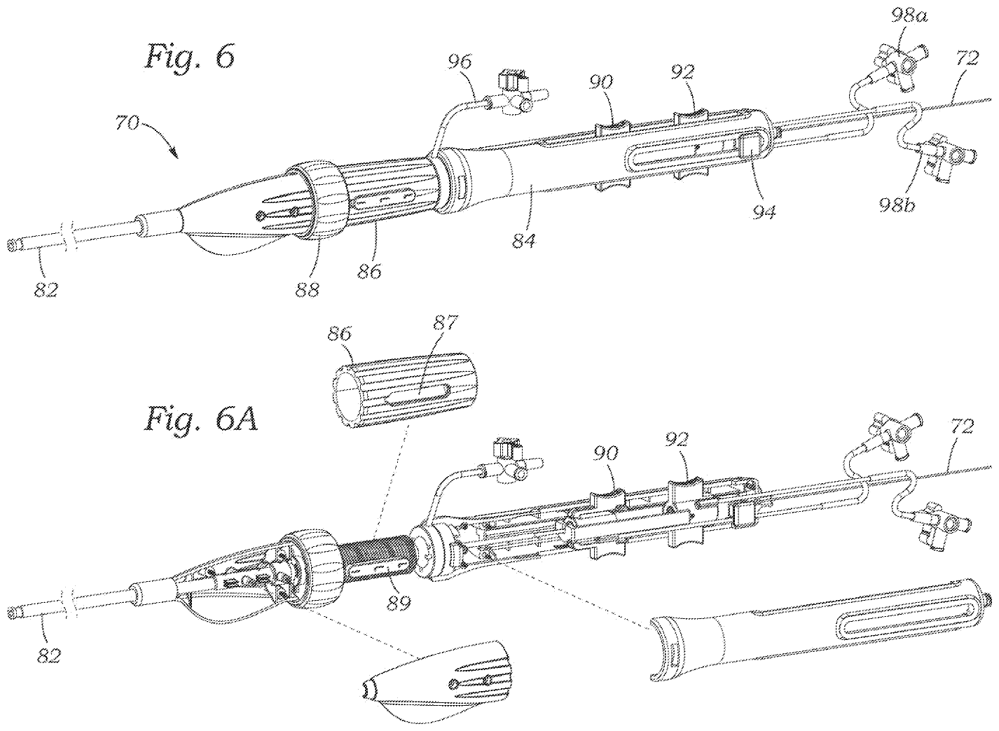

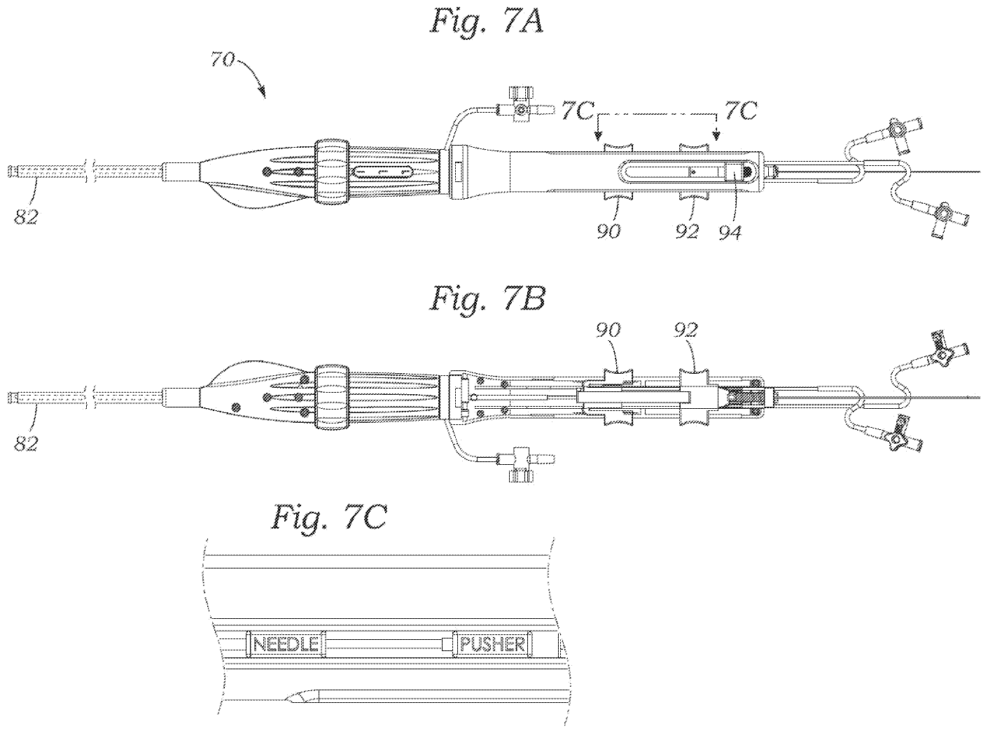

FIG. 6 is a broken perspective view of an exemplary active puncturing tool 70 of the present application, and FIG. 6A shows several outer covers exploded therefrom to expose inner workings. FIGS. 7A and 7B are similar elevational views thereof.

The active puncturing tool 70 generally comprises a proximal control handle 84 from which the flexible sheath 82 extends distally. The rear end of the puncturing needle 72 is seen projecting from a rear end of the control handle 84. The needle 72 is shown extending a greater distance than would be normal for the purpose of illustration. The needle 72 is hollow and extends along the length of the flexible sheath 82 from a location near the distal end thereof and proximally through the control handle 84.

The control handle 84 has a sleeve 86 and rotation ring 88 toward its distal end which actuate a steering mechanism for bending the flexible sheath 82. Although it will not be described in great detail, the sleeve 86 rotates about a longitudinal axis of the tool 70 and has an elongated slot 87 into which fits a similarly sized rail 89. The rail 89 is coupled to a rotation mechanism configured to displace a pull wire (not shown) extending down the length and to the end of the flexible sheath 82. By fastening the pull wire to one side of the distal tip of the flexible sheath 82, the distal tip may be deflected toward that side.

A gripping portion of the control handle 84 houses a pair of sliders 90, 92 which linearly displace, respectively, the puncturing needle 72 and the expandable anchor 56 through the needle. As will be described below, the two sliders 90, 92 are coupled to move axially together within a hollow housing of the control handle 84. Additionally, the proximal slider 92 is adapted to move axially relative to the distal slider 90 upon actuation of a locking tab 94. In particular, the hollow needle 72 extends to the end of the flexible sheath 82 and the distal slider 90 engages the needle for axial movement. The proximal slider 92 engages a pusher for the expandable anchor 56 so that the anchor may be displaced through the hollow needle 72. Concentric spaces are thus formed between the several concentrically-arranged tubes extending along the sheath 82.

A distal fluid port 96, and a pair of proximal fluid ports 98a, 98b are connected to different chambers within handle 84 for aspiration or injecting contrast medium. For example, one of the proximal fluid ports 98a, 98b may be used to inject contrast medium into the needle 72 so that it may be seen on fluoroscope in conjunction with the EKG locating method. Alternatively, each of the fluid ports 96, 98a, 98b may be used to withdrawing fluid or gas from concentric spaces within the system, including a space between the sheath 82 and needle 72, and a space between the needle 72 and cardiac anchor 56.

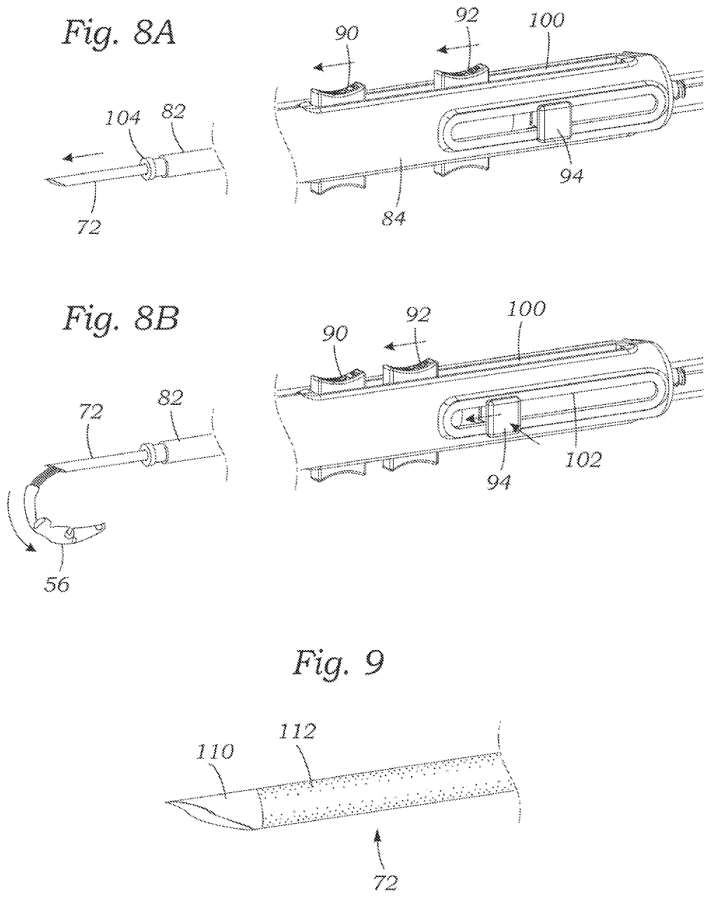

FIGS. 8A and 8B are partial views of the active puncturing tool 70 in several stages of deployment. Initially, the needle 72 may be displaced past a distal tip 104 of the flexible sheath 82. As explained, the two sliders 90, 92 translate together axially in a distal direction to displace the needle 72. The proximal slider 92 controls movement of the anchor 56, which is positioned within a distal region of the needle 72 and moves therewith. The locking tab 94 moves with the proximal slider 92. The two sliders 90, 92 each may have a pair of opposed ergonomic finger tabs which extend outward through longitudinal slots 100 in the top and bottom of the housing of the control handle 84, and the locking tab 94 is connected to move with the proximal slider 92 through a longitudinal slot 92 in the lateral side of housing. The ergonomic finger tabs are preferably labeled NEEDLE and PUSHER (for the anchor 56) for convenience, as seen in FIG. 7C.

FIG. 8B indicates inward actuation of the locking tab 92 so that it and the proximal slider 92 may be displaced in a proximal direction relative to the distal slider 90. As mentioned, the proximal slider 92 is coupled to displace the anchor 56 relative to the needle 72, and the anchor is thus shown being expelled from the needle 72 and curling into its deployed configuration.

It should again be noted that the puncturing needle 72 is configured as an electrode to transmit electrical cardiac pulses from within the body to an EKG monitor. FIG. 9 is an enlarged perspective view of the sharp distal tip 110 of the puncturing needle 72 showing insulation 112 thereon. More particularly, the needle 72 is desirably coated with an insulating material along its entire length except for at the distal tip 110, and of course at its proximal end where it connects with the aforementioned EKG lead. This focuses the region at which conductive signals are sensed by the needle 72, and thus increases accuracy of the needle location procedure. In one embodiment, only the distal 1-2 mm of the tip 110 of the needle 72 is exposed and can conduct electrical signals.

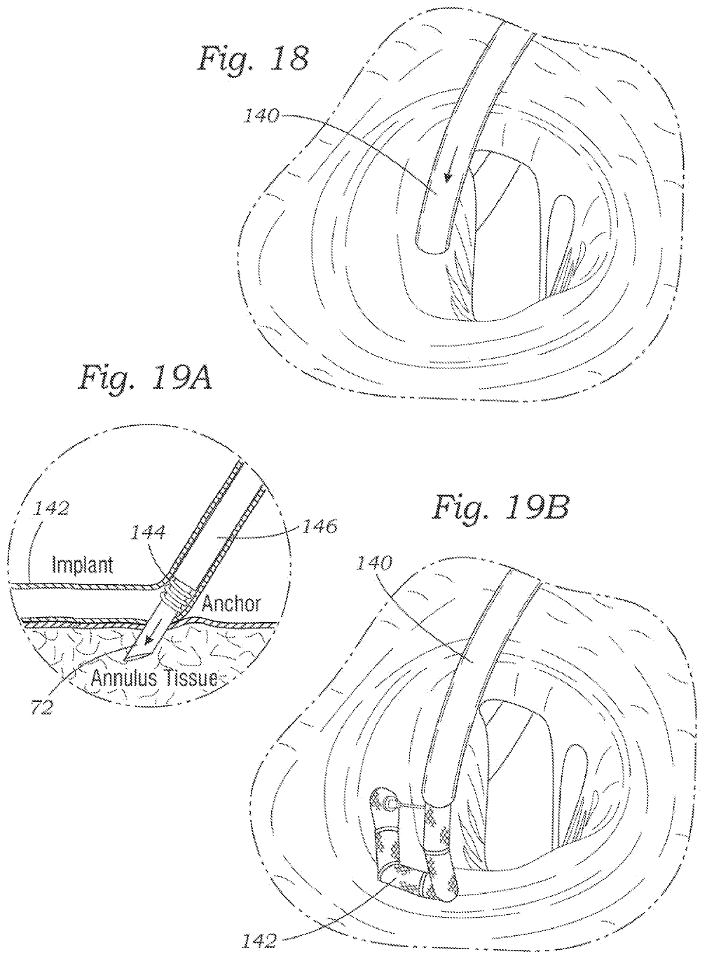

The puncturing needle 72 is but one of a number of potential probes or locating tips that may be utilized in the practice of the present locating and deploying system and method. The hollow needle 72 is particularly useful as the anchor 56 may then be deployed directly from its distal tip 110. However, a solid probe may also be used which then acts as a guidewire of sorts for delivery of an anchor over it, such as described below with respect to the embodiment shown in FIGS. 18-19. Similarly, the needle 72 or other probe may be retracted first after which an anchor may be advanced a known distance to the last location of the tip of the needle/probe. In this respect, the terms electrode probe or electrode needle are synonymous and may be used to generically define the locating tip that passes in and out of cardiac tissue and conducts electrical signals.

Further, the flexible electrode probe or electrode needle is made to conduct electrical pulses from within the heart, in or out of tissue. Such pulses are typically measured in voltage changes, and thus the probe or needle is made of an electrically conductive material such as copper or a ferromagnetic alloy. In one embodiment, the probe or needle is formed of conductive Stainless Steel, such as 304 SS alloy with 8% chromium and 8% nickel.

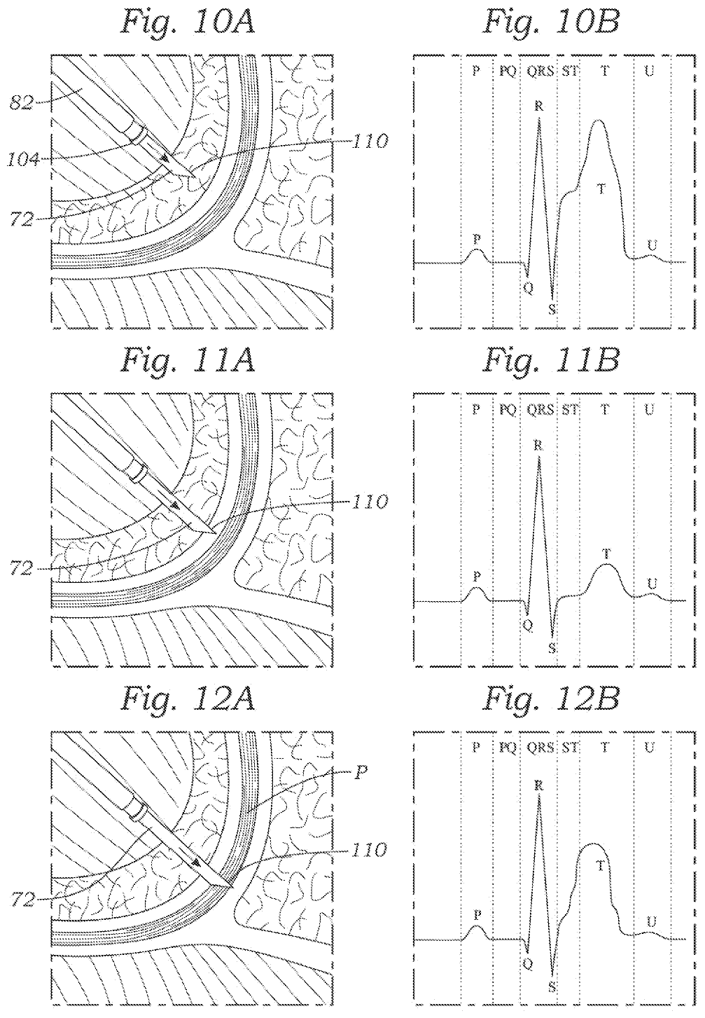

FIGS. 10A/10B, 11A/11B and 12A/12B show gradual advancement of the puncturing needle through the heart wall in a trans-pericardial approach alongside complementary images of a readout of an EKG system having one electrode connected to the puncturing needle 72. Initially, as explained above, the flexible sheath 82 is advanced through the access pathway (e.g., femoral vein) through the tricuspid valve and into the right ventricle. The distal tip 104 of the sheath 82 may be formed as an enlarged echogenic ring, for example, which is highly visible using echocardiography. The sheath 82 is halted in proximity with an inner wall of the right ventricle, and the sharp distal tip 110 of the needle 72 advanced into the myocardium, as in FIG. 10A. This results in an increase in the S-T segment of the EKG trace, as seen in FIG. 10B.

Subsequently, further advancement of the distal tip 110 through the myocardium and into the space within the pericardial sac as seen in FIG. 11A changes the character of the EKG readout. That is, the conductive tip 110 is now in a cavity rather than in tissue, and the EKG trace reserves to a more normal character with a diminished S-T segment, as indicated in FIG. 11B.

Finally, FIG. 12A shows the needle 72 advanced until the distal tip 110 is within the pericardial sac P, which again alters the EKG readout. Namely, the S-T segment is once again elevated above normal. EKG monitors typically also display numerical values of the various peaks and troughs of the cyclic trace, and thus the change in magnitude of the S-T segment can easily be seen.

In combination with one or both of fluoroscopy and echo, utilization of the active puncturing tool 70 greatly enhances the ability to quickly and accurately locate the distal tip 110 of the needle 72 for subsequent deployment of the anchor 56. With this additional indication, it becomes readily apparent in the trans-pericardial approach, when the needle 72 has transitioned from the RV space through to the pericardial space and further through to the chest cavity. During each of these steps, the operator will have a live real-time readout on the EKG monitor with a signal trace that is specific to what the needle 72 is in direct contact with. For example, as mentioned, transitioning from the RV free space to contact with the RV myocardium results in a significant S-T segment elevation in the EKG, indicating with great confidence that the needle tip is in contact with the myocardium. Once the needle is pushed through to the free space of the chest cavity, this S-T segment elevation disappears, and the operator can confirm they are in a free space to deploy the anchor. Incorporating this additional sensor input has reduced the time required to confirm successful puncture and dramatically reduced intra-procedural complications during animal studies that have been conducted.

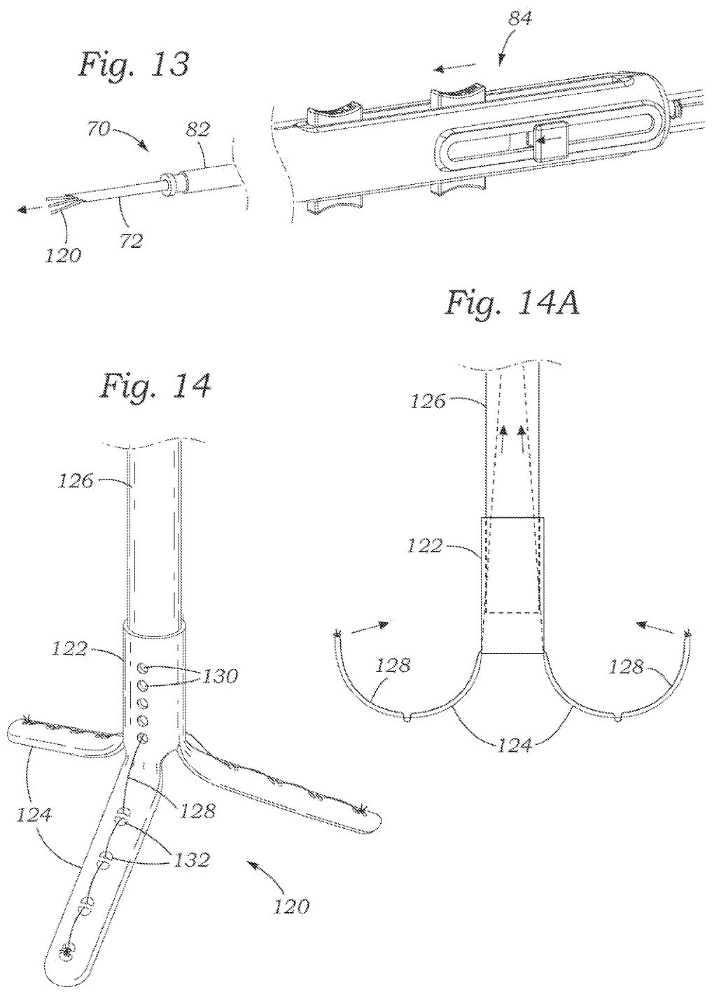

FIG. 13 is a perspective view of the active puncturing tool 70 incorporating an alternative grappling-hook style anchor 120 emerging from the needle 72. FIG. 14 is a perspective view of the alternative anchor 120 which comprises a proximal tubular barrel 122 and a plurality of individual prongs or tines 124 on the distal end thereof. The tubular barrel 122 may be mounted to the distal end of a hollow flexible shaft or tether 126 used to couple the anchor 120 to a regurgitation reduction spacer or other implant.

A plurality of retention sutures or filaments 128 extend through the tether 126 and proximally to the control handle 84. Each of the retention sutures 128 emerges radially outward through one of a plurality of holes 130 in the tubular barrel 122 and extends along primarily on the outside of one of the tines 124 to be secured at a distal tip thereof. In one embodiment, a series of cleats 132 are formed along each tine 24 and the retention suture 128 is woven through the cleats. For example, each of the cleats 132 may be formed by a pair of openings separated by a bridge such that the suture 128 passes down and then back up again through the thickness of each tine. The sutures 128 extend along radially outer sides of the tines 124 so as to exert an outward force thereon when pulled.

The anchor 120 is shown with three tines 124 evenly distributed about a longitudinal axis of the tubular barrel 122 (i.e., 120° apart) which are relatively wider than thick and have rounded distal tips. Of course, there may be more than three tines 124 and the distal tips may be more pointed. The tines 124 may be formed from an extension of the tubular barrel 122 and thus have convex outer surfaces, though they also may be flattened. Preferably, the anchor 120 is formed by laser cutting a tubular blank of Nitinol, and shape set (i.e., heat treated) so that the tines 124 splay outward when relaxed. The tines 124 thus having an undeployed configuration in which they are constrained substantially longitudinally within the needle 72, extending in a distal direction, and a deployed configuration wherein the tines are advanced distally from within the needle 72 and splay radially outward. The tines 124 each have a radially outward spring bias so as to separate in the deployed configuration toward a relaxed configuration with their free ends extending generally in a proximal direction.

The retention sutures 128 are utilized to apply tensile force along and to the distal tip of each of the tines 124. With reference to the sectional view of FIG. 14A, activation of a retention system is illustrated. Namely, tensile forces in a proximal direction on the retention sutures 128 are transmitted on each of the tines to their distal tips. This causes the distal tips to curl upward or in a proximal direction. To facilitate this behavior, each of the tines 124 may be performed with a proximal curve in a relaxed configuration such that they are straightened out when initially located within the needle 72. Once embedded in tissue, the tines 124 begin to curl back in a proximal direction which may be aided by tension on the retention sutures 128.

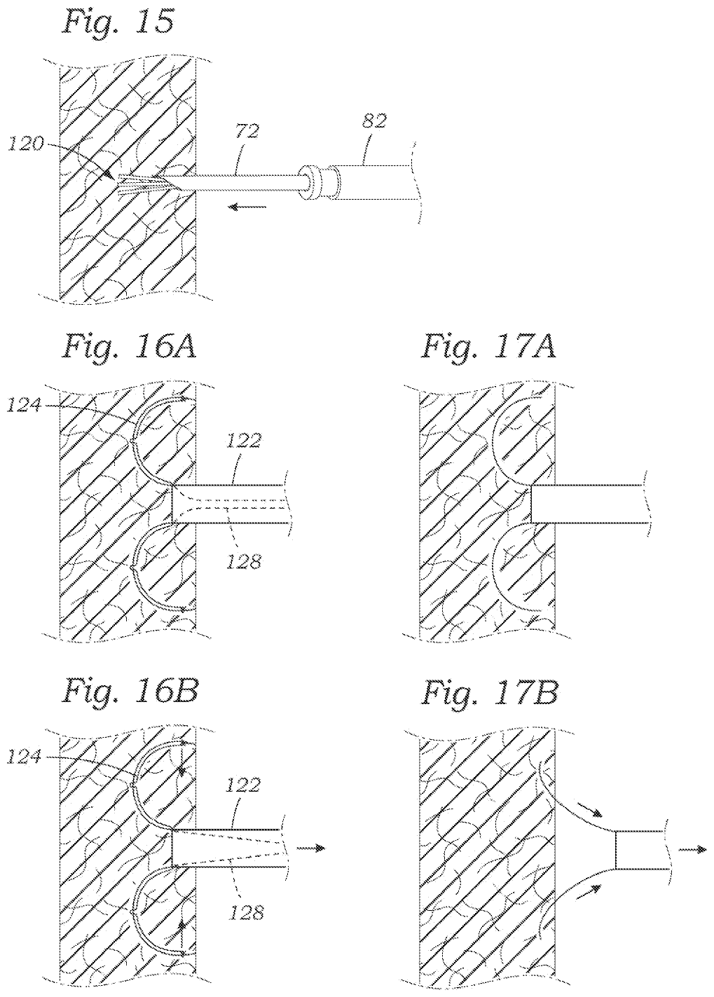

FIG. 15 is a view of the puncturing needle 72 embedded in tissue with the alternative anchor 120 of FIG. 14 advancing into the tissue. The positive location of the distal tip of the puncturing needle 72 in the tissue is once again desirably confirmed by using the connected EKG monitor, and possibly one or both of fluoroscopy and echo. At this point, the tines of the anchor 120 begin to splay outward and curl back upon themselves, so as to reach the fully deployed position shown in FIG. 16A.

Subsequently, as shown in FIG. 16B, tension on the retention sutures 128 pulls the distal tips of the tines 124 inward as shown by arrows, which helps retain the anchor 120 in the tissue, effectively directly resisting the tendency of the anchor to pull out of the tissue. That is, any proximal forces applied to the tubular barrel 122 ordinarily would tend to pull the flexible tines from the tissue. For example, FIG. 17A is a cross-sectional view illustrating a conventional anchor embedded into tissue, and FIG. 17B shows the potential for withdrawal from within the tissue from tensile forces. That is, without the retaining action of the sutures 128, the tines of a conventional anchor will tend to straighten and pull from within the tissue from proximal forces on the tether. A desirable tension can thus be established within the retaining sutures 128 after which the sutures are tied off to a proximately located part of the implant, such as the regurgitation reduction spacer described above.

As mentioned, the needle location system and method are useful for a number of different cardiac procedures. For example, FIG. 18 is a schematic view showing advancement of an annuloplasty band deployment sheath 140 into proximity with a heart valve annulus. FIG. 19A is a sectional view illustrating a step of deployment of the annuloplasty band 142 using the sheath 140 of FIG. 18. The sheath 140 incorporates an active puncturing needle 72 as described above to accurately position the needle. The needle 72 passes through a coiled anchor 144 housed within a larger delivery tube 146 that extends through the hollow band 142, with the concentric assembly advancing to the annulus through the deployment sheath 140.

FIG. 19B is a perspective view after a portion of the annuloplasty band 142 has been implanted at the annulus. The deployment system embeds a series of anchors 144 at spaced locations in the annulus tissue, sequentially securing the annuloplasty band 142 around the annulus. The system is similar to one sold under the name Cardioband Mitral and Tricuspid Valve Reconstruction System by Edwards Lifesciences of Irvine, CA, with the addition of the active puncturing needle 72. Although not shown, a lead of an EKG system is connected to the needle 72 which is insulated except for a conductive tip and functions in the manner as described above. In contrast to the earlier-described systems, the needle passes through the middle of the anchors 144 instead of vice versa. A concentric pusher (not shown) within the delivery tube 146 advances and rotates the anchors 144 relative to the needle 72 one-by-one. The needle 72 retracts into the delivery tube 146 after each anchor 144 is deployed and is subsequently advanced at a different location to ensure the next anchor is embedded at the proper tissue depth.

While the foregoing is a complete description of the preferred embodiments of the invention, various alternatives, modifications, and equivalents may be used. Moreover, it will be obvious that certain other modifications may be practiced within the scope of the appended claims.

What is claimed is:

1. A system for delivering and deploying a tissue anchor in a person, comprising:
an active puncturing tool including:
a proximal control handle with a flexible sheath extending distally therefrom,
a flexible puncturing needle extending through the sheath and linearly movable therein to a position beyond a distal tip of the sheath, the needle having a distal tip, and
a tissue anchor movable through the sheath to a position beyond the distal tip of the needle; and
at least one lead in electrical contact with the needle for recording an electrical signal from the heart of the person.

2. The system of claim 1, wherein the system is for delivering and deploying cardiac anchors, the flexible puncturing needle is electrically insulated except at the distal tip, and the at least one lead is a lead of an EKG system connected so that one lead is in electrical contact with a proximal end of the needle.

3. The system of claim 1, wherein the tissue anchor is an expandable disk-shaped anchor configured to abut tissue.

4. The system of claim 1, wherein the tissue anchor is configured to embed in tissue and comprises:
a tubular barrel defining a longitudinal axis having a plurality of distally-extending tines configured to be embedded into tissue, the tines being biased toward a relaxed configuration where the tines splay radially outward from the axis;
a flexible proximal shaft connected to the barrel; and
a plurality of sutures each connected to one of the tines and extending proximally through the shaft, each tine extending outward from the barrel and along a respective tine to be fastened at a distal tip thereof, wherein tension on the sutures helps prevent the tines from bending toward the axis upon application of proximal forces on the anchor tending to pull the anchor from within tissue.

5. The system of claim 4, wherein each of the tines is formed as a laser-cut portion of a tube that also forms the tubular barrel, and each tine has a plurality of cleats along its length through which the sutures pass before reaching the distal tip.

6. The system of claim 1, wherein the control handle has a first slider movable thereon configured to axially displace the needle relative to the sheath, and the proximal control handle has a second slider movable thereon configured to axially displace the tissue anchor relative to the needle.

7. The system of claim 6, wherein the first and second sliders are coupled for common movement, and further including a lock configured to be released to permit the second slider to move with respect to the first slider.

8. The system of claim 6, wherein each of the first and second sliders includes an outer finger tab labeled with an indicator of the respective function of each.

9. The system of claim 1, wherein the control handle further includes an actuator for angling a tip of the sheath.

10. The system of claim 1, wherein the needle is hollow and the tissue anchor is positioned within and deployable from within the needle.

11. The system of claim 10, wherein the control handle further includes a plurality of fluid ports connected thereto for introducing or withdrawing fluid or gas from concentric spaces within the system, including a space between the sheath and needle, and a space between the needle and tissue anchor.

12. A system for delivering and deploying a cardiac tissue anchor in a person, comprising:
an active puncturing tool including:
a proximal control handle with a flexible sheath extending distally therefrom,
a flexible puncturing needle extending through and beyond the sheath, the needle having a distal tip, and
a tissue anchor movable through the sheath to a position beyond the distal tip of the needle; and
at least one lead of an EKG system in electrical contact with the needle for recording an electrical signal from the heart of the person.

13. The system of claim 12, wherein the flexible puncturing needle is electrically insulated except at the distal tip, and the lead is in electrical contact with a proximal end of the needle, the EKG being a 5-lead EKG.

14. The system of claim 12, wherein the tissue anchor is an expandable disk-shaped anchor configured to abut tissue.

15. The system of claim 12, wherein the tissue anchor is configured to embed in tissue and comprises:
a tubular barrel defining a longitudinal axis having a plurality of distally-extending tines configured to be embedded into tissue, the tines being biased toward a relaxed configuration where the tines splay radially outward from the axis;
a flexible proximal shaft connected to the barrel; and
a plurality of sutures each connected to one of the tines and extending proximally through the shaft, each tine extending outward from the barrel and along a respective tine to be fastened at a distal tip thereof, wherein tension on the sutures helps prevent the tines from bending toward the axis upon application of proximal forces on the anchor tending to pull the anchor from within tissue.

16. The system of claim 12, wherein the flexible puncturing needle is linearly movable within the sheath to a position beyond a distal tip of the sheath.

17. The system of claim 16, wherein the control handle has a first slider movable thereon configured to axially displace the needle relative to the sheath, and the proximal control handle has a second slider movable thereon configured to axially displace the tissue anchor relative to the needle.

18. The system of claim 17, wherein the first and second sliders are coupled for common movement, and further including a lock configured to be released to permit the second slider to move with respect to the first slider.

19. The system of claim 17, wherein each of the first and second sliders includes an outer finger tab labeled with an indicator of the respective function of each.

20. The system of claim 12, wherein the control handle further includes an actuator for angling a tip of the sheath.

21. The system of claim 12, wherein the needle is hollow and the tissue anchor is positioned within and deployable from within the needle.

22. The system of claim 21, wherein the control handle further includes a plurality of fluid ports connected thereto for introducing or withdrawing fluid or gas from concentric spaces within the system, including a space between the sheath and needle, and a space between the needle and tissue anchor.
